(19) United States
(12) Patent Application Publication
(10) Pub. No.: US 2015/0190331 A1
(43) Pub. Date: Jul. 9, 2015

(54) NAIL LACQUER COMPOSITION WITH HYPOSENSITIVITY

(71) Applicant: Pakaly Enterprise Co., Ltd., New Taipei City (TW)

(72) Inventors: Pao-Min CHANG, New Taipei City (TW); Chin-Hao CHANG, New Taipei City (TW); Yung-Chin CHANG, New Taipei City (TW)

(73) Assignee: Pakaly Enterprise Co., Ltd., New Taipei City (TW)

(21) Appl. No.: 14/151,256

(22) Filed: Jan. 9, 2014

(51) Int. Cl. A61K 8/81; A61K 8/04; A61Q 3/02

(52) U.S. Cl. CPC A61K 8/8152; A61Q 3/02; A61K 8/042

(57) ABSTRACT

The present invention provides a gel polish composition characterized by containing no irritating reactive (meth)acrylate monomer, such as hydroxyethylmethacrylate (HEMA) and ethyl methacrylate (EMA). In the present invention, the gel polish composition comprises at least an aliphatic urethane acrylate oligomer, a polyester acrylate oligomer, a low-irritating acrylate monomer, and a photoinitiator. The gel polish composition possesses low or no irritating effects to skin, exhibits excellent adhesiveness to nails, and is stable in long-term storage at ordinary shelf conditions.

### BACKGROUND OF THE INVENTION

1. Technical Field of the Invention

The present invention relates to a nail coating composition, especially a gel lacquer composition, without containing irritating reactive (meth)acrylate monomer, such as hydroxyethylmethacrylate (HEMA) and ethyl methacrylate (EMA). In particular, the present invention relates to a nail coating composition comprising aliphatic urethane acrylate oligomer, polyester acrylate oligomer, low-irritating acrylate monomer, and photoinitiator.

2. Background

Nail polish is a lacquer applied to human finger or toe nails to decorate and protect the nail plate. There are three types of nail polish: Base Coat, which is a clear or milky colored polish formula that is used specifically before applying nail polish to the nail. The purpose of it is to strengthen nails, restore moisture to the nail, and/or help polish adhere to the nail. Top Coat is a clear colored polish formula that is used specifically after applying nail polish to the nail. It forms a hardened barrier for the nail to prevent chipping, scratching and peeling. Gel nail lacquer is a polish that is cured under a UV light, and has become a popular way for women to enhance the strength of their natural nails and to minimize the frequency with which they need a manicure.

Gel polish is a long-lasting type of nail polish. It is painted on the nail like a regular polish, and does not dry until it is “set” under an ultraviolet or LED lamp. Gel polish compositions can last up to two weeks without chipping, saving precious time in reapplying nail polish. While regular nail polish formulas typically last 2-7 days before chipping, gel polish lasts around two weeks on most. However, gel polish is more difficult to remove than regular nail polish; it is usually scraped off after soaking the nails in acetone for 5-10 minutes. It is not possible to remove gel polish with non-acetone nail polish remover. The most common type of nail polish remover contains the volatile organic compound acetone. It is powerful and effective, but is harsh on skin and nails, which makes them more brittle. Less toxic to children is ethyl acetate, the active ingredient in non-acetone nail polish removers, which also often contain isopropyl alcohol. Ethyl acetate is generally the solvent in nail polish itself.

U.S. Pat. No. 8,541,482 disclosed a liquid composition comprising reactive monomers, and/or oligomers, and/or polymers which provides the polymerized composition increased adhesiveness and removability. In 482's disclosure, such reactive monomers, and/or oligomers, and/or polymers may be a (meth)acrylate, selected from the group consisting of hydroxyethylmethacrylate (HEMA), hydroxypropylmethacrylate (HPMA), ethyl methacrylate (EMA), tetrahydrofurfuryl methacrylate (THFMA), pyromellitic dianhydride di(meth)acrylate, pyromellitic dianhydride glyceryl dimethacrylate, pyromellitic dimethacrylate, methacroyloxyethyl maleate, 2-hydroxyethyl methacrylate/suceinate, 1,3-glycerol dimethacrylate/succinate adduct, phthalic acid monoethyl methacrylate, acetoacetoxy ethyl methacylate (AAEMA), and mixtures thereof.

However, Methylmethacrylate is a known skin irritant and sensitizer. It possesses a strong and unpleasant odor and its vapors are toxic and irritating to the eyes. In guinea pigs, it is known that HEMA, Isopropylidenediphenyl Bisglycidyl Methacrylate, Lauryl Methacrylate, and Trimethylolpropane Trimethacrylate are strong sensitizers. Butyl Methacrylate, Cyclohexyl Methacrylate, Hexyl Methacrylate, and Urethane Methacrylate are moderate sensitizers; and Hydroxypropyl Methacrylate is a weak sensitizer. However, PEG-4 Dimethacrylate and Triethylene Glycol Dimethacrylate are not sensitizers. See international Journal of Toxicology 24(Suppl 5): 53-100, 2005.

### SUMMARY OF INVENTION

Therefore, it is needed to developed a gel lacquer composition, containing no irritating reactive (meth)acrylate monomer, possessing excellent adhesiveness, and being easy to remove with a wooden or metal stick.

Accordingly, in one aspect, the present invention relates to a gel lacquer composition, comprising aliphatic urethane acrylate oligomer, polyester acrylate oligomer, low-irritant acrylate monomer, and photoinitiator. In certain embodiments of the invention, the gel lacquer composition comprises 20˜70% of aliphatic urethane acrylate oligomer, 10˜50% of polyester acrylate oligomer, 10˜50% of low-irritant acrylate monomer, and 2˜11% of photoinitiator.

In some embodiments, the gel lacquer composition further comprises an aromatic urethane acrylate oligomer, in amount of 2˜10%, when the content of concentrated colorants is more than 6%, by weight.

Preferably, the aliphatic urethane acrylate oligomer has a molecular weight of 800˜25,000 Dalton, with 2 to 15 acrylic functional groups. The molecular weight of the optional aromatic urethane acrylate oligomer is preferably 800˜45,000 Dalton, with 2 to 6 acrylic functional groups.

The polyester acrylate oligomer used as a resin for production of paints and coatings usually has a molecular weight of 800˜15,000 Dalton, with 2 to 6 acrylic functional groups. It is preferable to further comprise a hyperbranched polyester acrylate in the gel lacquer composition to enhance curing rate. The content of the hyperbranched polyester acrylate may be 2˜20% by weight of the total composition. The hyperbranched polyester acrylate preferably has a molecular weight of 800˜20,000 Dalton, with 8 to 18 acrylic functional groups, and is branched to form a spherical or dendritic shape.

As one character of the present invention, irritating reactive (meth)acrylate monomer is not included in the gel lacquer composition. Therefore, low- or non-irritating acrylate monomers are suitably used in the present invention. Examples of such acrylate monomer include: triethylene glycol dimethacrylate (TEGDMA), ethoxylated trimethylolpropane tricarylate (TMP3EOTA), isobornyl acrylate (IBOA), propoxylated glyceryl triaerylate (G3.5POTA), cyclic trimthylolpropane formal acrylate (CTFA), ethoxylated phenoxyl acrylate, 2-(2-ethoxyethoxy) ethyl acrylate, cyclic trimethylolpropane formal acrylate, ethoxylated bisphenol-A (meth)diacrylate (2˜15EO), polyethylene glycol (200˜600) (meth)diacrylate, and ethoxylated trimethylolpropane(meth)triacrylate (3˜15EO). It is preferable to use a combination of TEGDMA and IBOA as the acrylate monomer in the gel lacquer composition. Preferably, the combination of TEGDMA and IBOA comprises 10˜40% by weight of TEGDMA and by weight of 10˜40% IBOA.

Optionally, a thiol monomer is incorporated in the gel lacquer composition for increasing the curing rate. The thiol monomer suitable used in the present invention includes the thiol monomer having 2˜4 mercapto functional groups, such as (3-mercaptobutylate) ester of pentaerithrytol. The content of the thiol monomer is less than 5%, preferably 1˜4.5%, by weight of the total composition. The suitable thiol monomers used in the present invention are described in US 2013/0146077 A1.

### BRIEF DESCRIPTION OF THE DRAWINGS

No figures appended.

### DETAILED DESCRIPTION OF THE INVENTION

The other characteristics and advantages of the present invention will be further illustrated and described in the following examples. The examples described herein are using for illustrations, not for limitations of the invention.

As one embodiment of this disclosure, the gel lacquer composition comprises: a oligomer combination consisted of 20˜70% of aliphatic urethane acrylate oligomer, 2˜10% of aromatic urethane acrylate oligomer, and 10˜50% of polyester acrylate oligomer, wherein the oligomer combination comprises 30˜70% of the gel lacquer composition; 10˜50% of a low-irritating acrylate monomer; 2˜11% of a photoinitiator; and a concentrated colorant containing pigments dispersed in the acrylate monomer. The gel lacquer composition may further comprise less than 5% of a thiol monomer for accelerating the solidification of gel lacquer.

In a preferable gel lacquer composition, the low-irritating acrylate monomer may be a combination consisted of 1040% TEGDMA and 10˜40% IBOA, the photoinitiator is TPO (diphenyl(2,4,6-trimethylbenzoyl)phosphine oxide) or TPO-L (Ethyl(2,4,6-trimethylbenzoyl)phenylphosphinate), and the thiol monomer is pentaerithrytol tetrakis(3-mercaptobutylate) ester.

Suitable pigments which can be incorporated into the concentrated colorant include barium, calcium and aluminum lakes, iron oxides, chromates, molybdates, cadmiums, metallic or mixed metallic oxides, talcs, carmine, titanium dioxide, chromium hydroxides, ferric ferrocyanide, ultramarines, titanium dioxide coated mica platelets, and/or bismuth oxychlorides, Preferred pigments include D&C Black No. 2, D&C Black No. 3, FD&C Blue No. 1, D&C Blue No. 4, D&C Brown No. 1, FD&C Green No. 3, D&C Green No. 5, D&C Green No. 6, D&C Green No, 8, D&C Orange No. 4, D&C Orange No. 5, D&C Orange No. 10, D&C Orange No. 11, FD&C Red No, 4, D&C Red No. 6, D&C Red No. 7, D&C Red No. 17, D&C Red No. 21, D&C Red No. 22, D&C Red No. 27, D&C Red No. 28, D&C Red No. 30. D&C Red No. 31, D&C Red No. 33, D&C Red No. 34, D&C Red No. 36, FD&C Red No. 40, D&C Violet No, 2, Ext. D&C Violet No, 2, FD&C Yellow No. 5, FD&C Yellow No. 6, D&C Yellow No. 7, Ext. D&C Yellow No. 7, D&C Yellow No. 8, D&C Yellow No. 10, D&C Yellow No. 11, as well as others listed on the FDA color additives website, and Annex IV of the Cosmetic Directive 76/768/EEC, Coloring Agents Permitted in Cosmetics.

The UV gel polish composition of the present invention may be made by any conventional method used in preparing nail polish compositions. Various exemplary formulations for producing embodiments of the UV gel polish composition are provided as follow. The resulting UV gel polish compositions are quickly cured after irradiation with UV Lamp (PHILIPS PL-L 18W*2) for 2 minutes, or with 6W LED Lamp for 1 minute. The cured gel polish has been tested in the Adhesion test, Stability test, and Touch-dry test. It is found that all of the UV gel polish composition of the present invention can confer the cured membrane with desired property of being smooth, dry, glossy, and well adhered to the nail.

Adhesion Test

The UV gel polish composition of the present invention is applied to the nails of 20 persons having various living conditions, and cured for 2 minutes under a standard 36W UV lamp. The coating may be kept on the nails of the persons who doesn't do housework frequently for 8˜14 days, and be removed from the nails of housekeepers after 3˜7 days.

Stability Test

The UV gel polish composition of the present invention is place in a nail polish bottle and put into a oven at 80° C. The uncured products remain mobile, and unchanged in viscosity or color after one week, indicating that the UV gel polish composition may have a shelf life of 1˜2 years at room temperature.

Touch-Dry Test

The gel polish composition of the present invention is applied to a glass slide and cured for 2 minutes under a standard 36W UV lamp. The UV-light cured gel polish coating is wiped with toilet paper. It is observed that there is no scraps of paper stick on the surface of the gel polish coating, and the surface remain smooth and glossy after wiping.

### Example 1

| | Amount |
|---|---|
| Components | (wt %) |
| Aliphatic urethane acrylate oligomer | 28.5 |
| Polyester acrylate oligomer | 10 |
| Chlorinated polyester resin | 5 |
| Hyperbranched polyester acrylate | 8 |
| Triethylene glycol dimethacrylate (TEGDMA) | 10 |
| Isobornyl acrylate (IBOA) | 20 |
| Propoxylated glyceryl triacrylate (G3.5POTA) | 5 |
| Thiol monomer | 4.5 |
| 1-Hydroxycyclohexyl phenyl methanone (Irgacure 184) | 6 |
| Diphenyl(2,4,6-trimethylbenzoyl)phosphine oxide | 3 |
| (Irgacure TPO) | |
| | 100 |

### Example 2

| | Amount |
|---|---|
| Components | (wt %) |
| Aliphatic urethane acrylate oligomer | 26 |
| Aromatic urethane acrylate oligomer | 15 |
| Polyester acrylate oligomer | 10 |
| Chlorinated polyester resin | 6 |
| Triethylene glycol dimethacrylate (TEGDMA) | 10 |
| Isobornyl acrylate (IBOA) | 10 |
| Propoxylated glyceryl triacrylate (G3.5POTA) | 10 |
| Thiol monomer | 4.5 |
| 1-Hydroxycyclohexyl phenyl methanone (Irgacure 184) | 5.5 |
| Diphenyl(2,4,6-trimethylbenzoyl)phosphine oxide | 3 |
| (Irgacure TPO) | |
| | 100 |

### Example 3

| | Amount |
|---|---|
| Components | (wt %) |
| Aliphatic urethane acrylate oligomer | 30 |
| Polyester acrylate oligomer | 10 |
| Triethylene glycol dimethacrylate (TEGDMA) | 12.5 |
| Ethoxylated trimethylolpropane tricarylate (TMP3EOTA) | 15 |
| Isobornyl acrylate (IBOA) | 16 |
| Propoxylated glyceryl triacrylate (G3.5POTA) | 7 |
| Thiol monomer | 4.5 |
| Diphenyl(2,4,6-trimethylbenzoyl)phosphine oxide | 5 |
| (Irgacure TPO) | |
| | 100 |

According to the present disclosure, a low- or non-irritating gel lacquer composition is provided for it does not contain any sensitizing reactive (meth)acrylate monomers and solvents. In addition to the low or non irritating effects to nail and skin, the present gel lacquer composition also possesses exhibits excellent adhesiveness. Therefore, it is convenient to apply an all-in-one IN gel polish composition on nail without pre-coating a Base Coat or Primer composition to enhance the adhesiveness of polish to the nail. The gel polish composition can provide a bright and smooth thin layer coating on the nails after cured under a UV light or LED lamp, so that it is no need to apply a further Top Coat composition to increase the surface brightness. Besides, the cured coating of the gel polish composition has good adhesion to the nails for 3 to 14 days, depending on the living condition of users.

1. A gel polish composition, comprising:
20˜70% by weight of an aliphatic urethane acrylate oligomer;
10˜50% by weight of a polyester acrylate oligomer;
10˜50% by weight of a low-irritating acrylate monomer; and
2˜11% by weight of a photoinitiator,
wherein the low-irritant acrylate monomer is selected from the group consisted of triethylene glycol dimethacrylate (TEGDMA), ethoxylated trimethylolpropane tricarylate (TMP3EOTA), isobornyl acrylate (IBOA), propoxylated glyceryl triacrylate (G3.5POTA), cyclic trimthylolpropane formal acrylate (CTFA), ethoxylated phenoxyl acrylate, 2-(2-ethoxyethoxyl) ethyl acrylate, cyclic trimethylolpropane formal acrylate, ethoxylated bisphenol-A (meth)diacrylate (2˜15EO), polyethylene glycol (200˜600) (meth)diacrylate, ethoxylated trimethylolpropane(meth)triacrylate (3˜15EO), and the combination thereof.

2. The gel polish composition of claim 1, wherein the acrylate monomer comprises a combination of TEGDMA and IBOA.

3. The gel polish composition of claim 2, wherein the acrylate monomer combination is consisted of 10˜40% TEGDMA and 10˜40% IBOA.

4. The gel polish composition of claim 1, wherein the aliphatic urethane acrylate oligomer has a molecular weight of 800˜25,000 Dalton, with 2 to 15 acrylic functional groups.

5. The gel polish composition of claim 1, further comprising 2˜10% by weight of an aromatic urethane acrylate oligomer.

6. The gel polish composition of claim 5, wherein the aromatic urethane acrylate oligomer has a molecular weight of 800˜25,000 Dalton, with 2 to 6 acrylic functional groups.

7. The gel polish composition of claim 1, further comprising 1˜4.5% by weight of a thiol monomer.

8. The gel polish composition of claim 7, wherein the thiol monomer includes (3-mercaptobutylate) ester of pentaerithrytol with 2˜4 mercapto functional groups.

9. The gel polish composition of claim 1, comprising a oligomer combination consisted of 20˜70% of aliphatic urethane acrylate oligomer, 2˜10% of aromatic urethane acrylate oligomer, and 10˜50% of polyester acrylate oligomer, wherein the oligomer combination comprises 30˜70% by weight of the gel lacquer composition; 10˜50% by weight of a low-irritating acrylate monomer; 2˜11% by weight of a photoinitiator; and 1˜4.5% by weight of a thiol monomer for accelerating solidification; and a concentrated colorant containing pigments dispersed in the acrylate monomer.

10. The gel polish composition of claim 7, wherein the low-irritating acrylate monomer is a combination of TEGDMA and IBOA; the photoinitiator is TPO (diphenyl(2,4,6-trimethylbenzoyl)phosphine oxide) or TPO-L (Ethyl(2,4,6-trimethylbenzoyl)phenylphosphinate); and the thiol monomer is pentaerithrytol tetrakis(3-mercaptobutylate) ester.

11. The gel polish composition of claim 10, wherein the acrylate monomer combination comprises 10˜40% by weight of TEGDMA and 10˜40% by weight of IBOA.
